(19) United States
(12) Patent Application Publication
(10) Pub. No.: US 2025/0120815 A1
(43) Pub. Date: Apr. 17, 2025

(54) IMPLANT FUSION DEVICE WITH ENHANCED OSTEOINDUCTIVITY

(71) Applicant: Spectrum Spine IP Holdings, Inc., Inlet Beach, FL (US)

(72) Inventor: James C. Robinson, Inlet Beach, FL (US)

(21) Appl. No.: 18/488,486

(22) Filed: Oct. 17, 2023

(51) Int. Cl. A61F 2/30; A61F 2/28; A61F 2/44

(52) U.S. Cl. CPC A61F 2/3094; A61F 2/4405; A61F 2002/2835; A61F 2002/30535; A61F 2002/3093; A61F 2002/3097

(57) ABSTRACT

An implant device has an improved osteoinductive feature to enhance new bone formation. The implant device has a body structure having a superior or first surface and an inferior or second surface and one or more facet features. One or more exterior and interior side surfaces extend between the superior and inferior surfaces. The one or more facet features extend from the internal side surfaces. Each of the facet features is inclined off parallel relative to the exterior side surface or off perpendicular relative to the superior and inferior surfaces at an angle which provides a surface to facilitate laser modification along the facet feature when a laser beam is oriented at an angle generally perpendicular to the load bearing inferior or superior surface.

### FIELD OF THE INVENTION

The present invention relates to an implant fusion device and a method of manufacturing an implant fusion device. More particularly an orthopedic or spinal implant configured to be implanted between adjacent vertebrae or within a gap in a bone or between bones, the device having internal and/or external facet or inclination and/or aperture features to facilitate laser engraving of the implant surfaces to enhance osteoinductivity.

### BACKGROUND OF THE INVENTION

Surgical implantation of interbody cages is typically used to provide support along the spinal column in cases where a portion of the patient's intervertebral anatomy has become weakened, diseased, or destroyed. Such support systems are also commonly used following a discectomy, where an intervertebral disc is surgically removed.

Most commonly, existing support systems typically operate by inhibiting normal movement between the adjacent vertebrae, thereby stabilizing these vertebrae at fixed positions relative to one another, with the mechanical body of the supporting structure providing the needed support along the patient's spinal column. Such supporting systems are typically made of stainless steel, titanium, titanium alloy, polymer (e.g., an organic polymer thermoplastic such as polyether ether ketone (PEEK)), carbon fiber, or ceramic and they are designed to permanently remain within the patient's body.

It is beneficial, in addition to fixation, to try to stimulate bone growth between the adjacent vertebrae. To do so, spine surgeons often use bone graft material in addition to fixation devices. Bone graft doesn't heal or fuse the spine immediately; instead, bone graft provides a foundation or scaffold for the patient's body to grow new bone. Bone graft can stimulate new bone production. When new bone grows and solidifies, fusion occurs. Although instrumentation (e.g., screws, rods) is often used for initial stabilization (post-operative), it is the healing of bone that welds vertebrae together to create long-term stability. There are two general types of bone grafts: real bone and bone graft substitutes. Real bone can come from the patient (autograft) or from a donor bone (allograft). Also used in these types of surgery are bone substitute, osteoinductive agents, stem cell products, bone morphogenic proteins, and bone cement.

There is a need for improved systems and methods for spinal fusion devices. Ideally, the spinal fusion implant device has features that facilitate new bone growth to achieve fusion of the adjacent vertebrae.

### DEFINITIONS

As used herein and in the claims:
3D printing, or additive manufacturing, is the construction of a three-dimensional object from a CAD model or a digital 3D model. The term “3D printing” can refer to a variety of processes in which material is deposited, joined or solidified under computer control to create a three-dimensional object, with material being added together, typically layer by layer.

The Angle of Incidence is the angle between a ray of light incident on a surface and the line perpendicular to the surface at the point of incidence, called normal. It is equal to the angle of reflection, which is the angle between the normal and the reflected ray at the point of incidence.

A facet refers to plane surfaces or any number of sides or aspects, as used herein, having at least two inclined intersecting surfaces projecting from a wall or planar surface.

A femtosecond laser is an infrared laser that emits bursts of laser energy at an extremely fast rate. A femtosecond laser has a pulse duration in the femtosecond range, or one quadrillionth of a second. A femtosecond laser is a laser that emits optical pulses with a duration well below 1 ps (→ultrashort pulses), i.e., in the domain of femtoseconds (1 fs=10-15 s). It thus also belongs to the category of ultrafast lasers or ultrashort pulse lasers (which also include picosecond lasers). Femtosecond (FS) laser is an infrared laser with a wavelength of 1053 nm.

Green lasers with the appropriate power required for industrial applications are usually frequency-doubled IR lasers with wavelengths between 515 nm and 532 nm. Their disadvantage is that they are—compared to the IR variants—less efficient, may lose power during a manufacturing process, and require expensive cooling and complex optical setups. The most common green lasers are actually infrared lasers, emitting natively 1064 nm light. There is a second crystal used to double the frequency, and half the wavelength down to 532 nm, providing green light. The infrared light should then be filtered out to get only green output.

Laser engraving and laser etching are both techniques used to create designs, but they differ in how they interact with materials and the range of materials they can be applied to. Laser engraving involves vaporizing the material, at least the top layer, allowing for deep marks on metal, wood, or plastic. Laser etching only melts and raises the topmost micro-layer without disturbing the internal structure.

Laser texturing is an emerging technology for generating surface functionalities on basis of optical, mechanical, or chemical properties. Taking benefit of laser sources with ultrashort (fs) pulse durations features outstanding precision of machining and negligible rims or burrs surrounding the laser-irradiation zone. Consequently, additional mechanical or chemical post-processing steps are usually not required for fs-laser surface texturing (fs-LST). This work aimed to provide a bridge between research in the field of tribology and laser materials processing.

Nanotechnology is the engineering of functional systems at the nanometer scale. This covers both current work and concepts that are more advanced. In its original sense, nanotechnology refers to the projected ability to construct items from the bottom up, using techniques and tools being developed today to make complete, high-performance products. Alternatively, nanostructure may be developed through subtractive processes.

Cortical or compact bone can be distinguished macroscopically from cancellous or trabecular bone. Cortical bone is a dense tissue that contains less than 10% soft tissue. Cancellous or spongy bone is made up of trabeculae, shaped as interconnected plates or rods and arced structures interspersed between voids in the mineral structure that contain blood cells in the marrow space which represents more than 75% of the cancellous bone volume.

Microtechnology/Laser Micro Machining: Although similar in concept to traditional machining operations, laser micro machining (laser micromachining) is capable of creating extremely small features—generally under 1 mm, and in some cases only a few microns in size—with a high degree of repeatability and without causing significant structural damage to the surrounding material.

Micron (μm) Microns, also known as micrometers (represented as μm) are a length of measurement equal to one millionth of a meter. (1,000um is equal to 1 mm.)

Nanotechnology: One nanometer (nm) is one billionth, or 10-9, of a meter. By comparison, typical carbon-carbon bond lengths, or the spacing between these atoms in a molecule, are in the range 0.12-0.15 nm, and a DNA double-helix has a diameter around 2 nm. On the other hand, the smallest cellular life-forms, the bacteria of the genus Mycoplasma, are around 200 nm in length. By convention, nanotechnology is taken as the scale range 1 to 100 nm following the definition used by the National Nanotechnology Initiative in the US. The lower limit is set by the size of atoms (hydrogen has the smallest atoms, which are approximately a quarter of a nm kinetic diameter) since nanotechnology must build its devices from atoms and molecules. The upper limit is more or less arbitrary but is around the size below which the phenomena not observed in larger structures start to become apparent and can be made use of in the nano device. These new phenomena make nanotechnology distinct from devices which are merely miniaturized versions of an equivalent macroscopic device; such devices are on a larger scale and come under the description of microtechnology.

“Prominent” standing out or projecting beyond a surface or line: protuberant.

Trabecular bone is a highly porous (typically 75-95%) form of bone tissue that is organized into a network of interconnected rods and plates and arcs called trabeculae which surround pores that are filled with cellular bone marrow.

### SUMMARY OF THE INVENTION

An implant device configured to be at least partially in contact with bone on implantation has an improved osteoinductive feature to enhance new bone formation. The implant device has a body structure and one or more facet features. The implant body structure has a superior or first surface and an inferior or second surface. Both surfaces are configured to be load bearing upon implantation. One or more exterior side surfaces and one or more interior side surfaces extend between the superior and inferior surfaces. The one or more facet features extend from the internal side surfaces. Each of the facet features is inclined off parallel relative to the exterior side surface or off perpendicular relative to the superior and inferior surfaces at an angle, which provides a surface to facilitate laser modification along the facet feature when a laser beam is oriented at an angle generally perpendicular to the load bearing inferior or superior surface.

The facet features are configured to facilitate laser modification to enhance osteoinductivity and promote bone ingrowth located in an internal or central bone graft space. At least a portion of the implant body structure is exposed to subtractive laser modification which creates a laser modified network having a random or non-random network of trenches, grooves or recesses having nano scale features or prominences for new bone growth formation to attach. The facet features are exposed to subtractive laser modification which creates a laser modified network having a random or non-random network of trenches, grooves or recesses having nano scale features or prominences for new bone growth formation to attach.

In one embodiment, the laser modified network includes superior and inferior surfaces in contact with bone on implantation as well as surfaces where new bone growth occurs on the internal surfaces and side surfaces of the device that help secure the device in place after bone fusion occurs. The facet features are inclined at any positive angle off from parallel relative to the side surface or off perpendicular relative to the superior and inferior surfaces.

In another embodiment, the implant body structure has one or more apertures in the side surface extending through to the internal or central bone graft space that allow for laser modification of interior surfaces across from the apertures.

The nano scale features are prominences projecting less than 200 nano meters from the one or more bone growth surfaces only visible through magnification of sub micron resolution exhibiting large surface areas relative to the size of the nano scale prominences configured to enhance and receive new bone growth providing the improved osteoinductive feature and variations of surface features resulting from the manufacturing process and the material composition of the implant device. An area of the one or more bone growth surfaces of the implant with the nano scale features or prominences having a surface area greater with no additional volume than the surface area of the non-laser engraved surfaces without the nano scale features or prominences and the nano scale features or prominences are less than 200 nano meters formed by subtraction laser engraving process on the surface of the bone growth surfaces of the implant.

The inferior and superior surfaces are exposed to subtractive laser modification which creates a laser modified network having a random or non-random network of trenches, grooves or recesses having nano scale features or prominences for new bone growth formation to attach. The laser modified network when having a non-random network is an organized pattern. The laser modified network is formed by emitting laser beams unobstructed to surfaces within the path of the laser beams. The laser modification can be laser engraving or laser etching.

A method of making an implant device configured to be at least partially in contact with bone on implantation having an improved osteoinductive feature to enhance new bone formation, has the steps of: providing an implant body structure, the implant body structure having a superior or first surface and an inferior or second surface, both surfaces configured to be load bearing upon implantation and one or more exterior side surfaces and one or more interior side surfaces extending between the superior and inferior surfaces; one or more facet features extending from the internal side surfaces, each of the facet features being inclined off parallel relative to the exterior side surface or off perpendicular relative to the superior and inferior surfaces at any positive angle which provides a surface to facilitate laser modification along the facet feature when a laser beam is oriented at an angle perpendicular to the load bearing inferior or superior surface; and wherein the implant body structure is stationary and a laser moves about the implant body structure to create a laser modified network or wherein a laser is stationary and the implant body structure moves relative to the laser to create a laser modified network.

The method of making a spinal implant device or orthopedic device or bone implant device further has the step of: laser modification on at least a portion of an exterior surface or surfaces of the implant body structure to create the laser modified network, the laser modified network creating new bone growth attachment features to enhance osteoinductivity of the spinal implant fusion device. The laser modified network is made into a network of features in either a random pattern or an organized pattern. The laser modification is formed by emitting laser beams unobstructed to the exterior surfaces and the interior side surfaces have one or more facet features.

The method of making a spinal implant device or orthopedic device or bone implant device further has the step of moving a laser about the implant body structure to create the laser modified network. The method of making a spinal implant device or orthopedic device or bone implant device further has the step of moving the implant body structure about a laser to create the laser modified network.

### BRIEF DESCRIPTION OF THE DRAWINGS

The invention will be described by way of example and with reference to the accompanying drawings in which:

FIG. 1A is a perspective view of an exemplary embodiment of the spinal implant fusion device of the present invention.

FIG. 1B is a perspective view of the implant device of FIG. 1A with laser engraving.

FIG. 2A is a superior perspective view of a second embodiment implant device of the present invention.

FIG. 2B is a superior perspective view of the second embodiment implant device of FIG. 2A with laser engraving.

FIG. 2C is a side perspective view of the second embodiment implant device of FIG. 2A.

FIG. 2D is a side perspective view of the second embodiment implant device of FIG. 2A with laser engraving.

FIG. 2E is a superior view of the second embodiment implant device of FIG. 2A with laser engraving.

FIG. 2F is a side view of the second embodiment implant device of FIG. 2A with laser engraving.

FIG. 2G is an opposite side view of the second embodiment implant device of FIG. 2F with laser engraving.

FIG. 3A is an inferior perspective view of a third embodiment implant device of the present invention.

FIG. 3B is an inferior perspective view of the third embodiment implant device of the present invention with laser engraving.

FIG. 4 is an exemplary laser engraving machine.

FIG. 5 is an enlarged view of a laser engraved surface made using the process of the present invention.

FIG. 6 is a magnification showing the nano features of the laser engraved surface made using the process of the present invention.

### DETAILED DESCRIPTION OF THE INVENTION

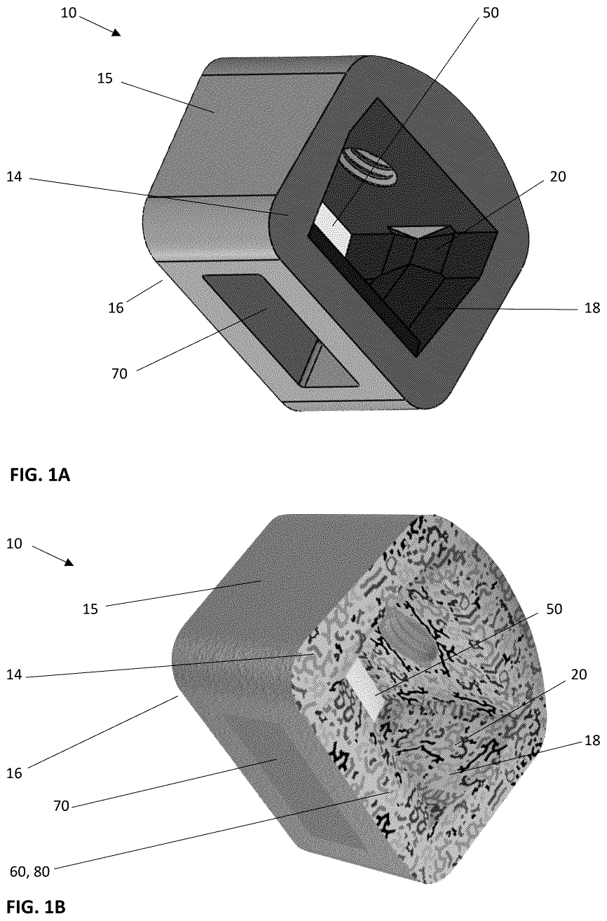

With reference to FIGS. 1A-3B, various embodiments of the implant fusion device 10 of the present invention are shown. The embodiments have common features or elements having the same reference numerals. The device 10 has facet features 20 that create a surface area that facilitates laser modification. Subtractive laser engraving creates a random or non-random network 80 of trenches, grooves or recesses having nano scale features 60 or prominences 60 for new bone growth formation to attach. These surface areas include surfaces in contact with bone on implantation as well as surfaces where new bone growth occurs on the internal surfaces 18 and side surfaces 15 of the device 10 that help secure the device in place after bone fusion occurs. One view in each set of figures shows the device without any laser modification, etching or carving so as to make the facets more readily apparent. The other views show the laser modification which, in the drawings, tends to conceal the facet features 20.

As shown in FIGS. 1A and 1B, exemplary implant device 10 is shown is shown as a cervical implant sized 9×12×14 mm with internal facet or inclination features 20 angled at an exemplary angle of 8 degrees that facilitate laser modification. The configuration of the implant device 10, as illustrated, has a first or superior external surface 14, a second or inferior external surface 16 and external side surfaces 15 that form the exterior surfaces of the implant 10. As shown, one of the exterior side surfaces has an opening or window forming an aperture 30 extending into and through the interior surface 18 allowing the laser beam to contact the interior surfaces 18 and facets 20. The first surface 14 and second surface 16 provide the implant fusion device 10 with surfaces that upon implantation between two adjacent vertebral bodies will support the bone structure of the adjacent vertebral bodies. These first and second surfaces 14, 16 are in direct contact with the bone structure of the adjacent vertebral bodies of the patient upon implantation of the device 10 for a procedure where an implant fusion device is being implanted to correct a degenerative condition or other condition in a patient. Internal surfaces 18 are formed with facet or inclination features 20. These facet features 20 provide surfaces to facilitate laser modification or engraving to enhance osteoinductivity and promote bone ingrowth located in the internal or central bone graft space 50. For most implants, these facet features 20 allow the laser which is oriented at a relatively perpendicular angle relative to the surface to be engraved as the laser beam contacts the facet feature surface. The inclination of the facets 20 provides a surface that projects outwardly from the interior wall of the implant body. The inclination can be any angle greater than zero relative to the interior surface or wall and the greater the angle means the more surface area is available for a laser beam to impinge at an incident angle off perpendicular. In the various embodiments shown in FIGS. 1A-3B, as shown, the facet features 20 were arbitrarily inclined at angles of less than 15 degrees off from parallel relative to the side surface 15 or off perpendicular relative to the superior and inferior surfaces 14, 16. This angle can vary between virtually any angle greater than zero, preferably between 5 and 30 degrees, more preferably less than 15 degrees. This allows the laser beam to create a laser modified network 80 along the inclined surface of the facet features 20 even though the laser beam is oriented perpendicular to the load bearing superior or inferior surfaces 14, 16.

As used herein, the facet features 20 are inclined relative to a perpendicular line extending between the superior and inferior surface in a range of 5 to 30 degrees, more particularly less than 15 degrees. Accordingly, assuming the implant device 10 has a flat superior or inferior surface, a flat plane would lie on these surfaces. From the flat plane, a perpendicular line would have the surface of the facet feature inclined off the perpendicular line at a positive angle, by way of example, an angle of 5 to 30 degrees, preferably less than 15 degrees. Wherein the perpendicular line is at or represents a 0 degree angle. Alternatively, if the exterior side surfaces were parallel, therefore perpendicular to the flat plane, then the facet feature would also be 5 to 30 degrees, preferably less than 15 degrees off parallel relative to the exterior side surface. In either case, one of ordinary skill would appreciate this creates a facet feature with a surface that can be laser engraved with the desired nano features. In the absence of facets 20, the ability to form laser modified networks on the interior surfaces was greatly limited, if not impossible, for a laser beam projecting perpendicular to the implant superior or inferior surface. This meant the beam had to be tilted in a variety of orientations taking much more time to create these osteoinductive features.

An important aspect of the facet features 20 is the internal or interior surfaces 18 of the implant body can be laser engraved on the inclined surface of the facet features 20 at a very fast rate while the laser beam is held substantially in a perpendicular orientation relative to the implant superior 14 or inferior surface 16. This means the laser beam can be passed over the device 10 without time consuming manipulation of the emitted laser beam to create the laser modified network 80 and nano scale features 60.

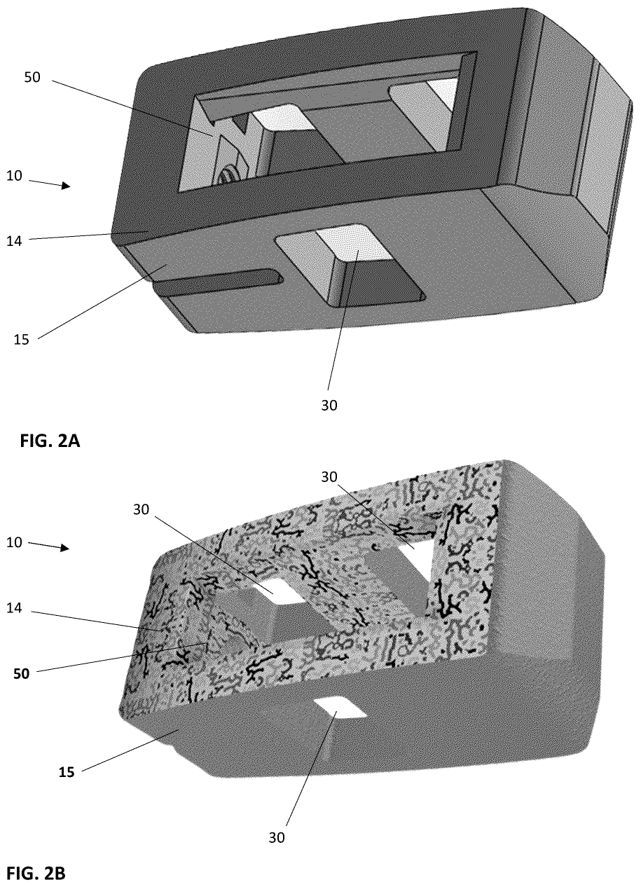

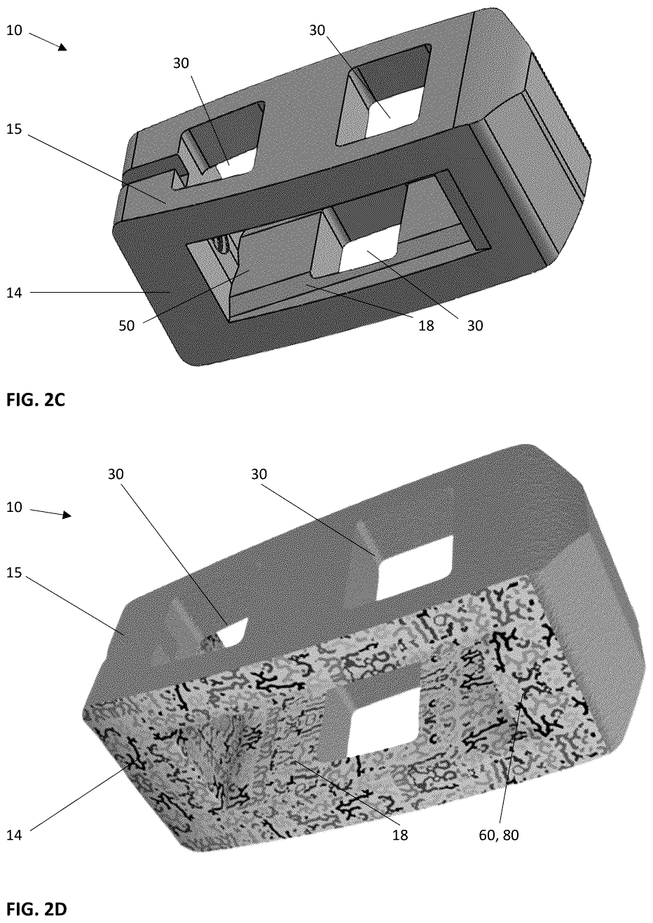

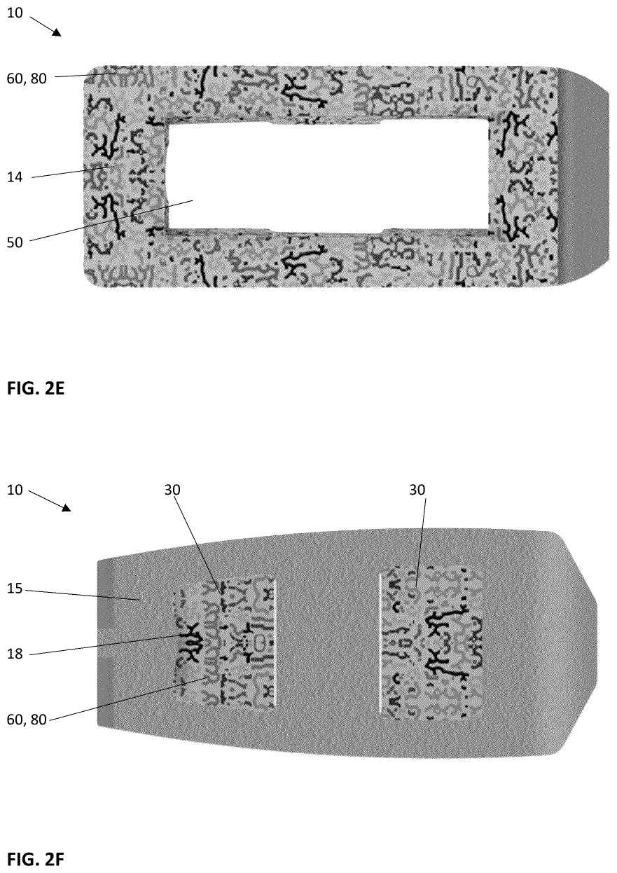

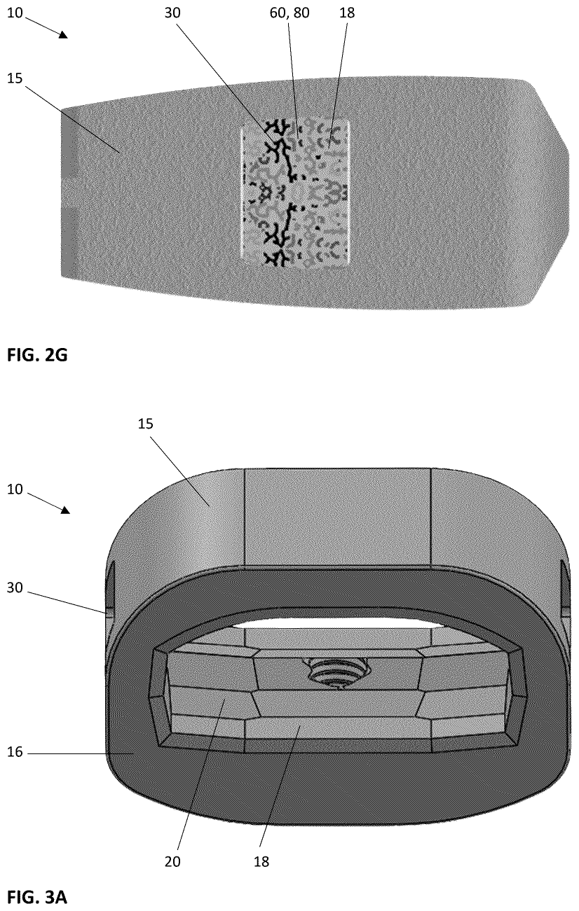

With reference to FIGS. 2A-2G, a second exemplary embodiment implant device is shown as a PLIF/TLIF sized 12×11×26 mm with facets 20 angled with a positive angle greater than zero, as illustrated at 12 degrees and having similar features of FIG. 1. This exemplary embodiment of the spinal implant fusion device 10 of the present invention is illustrated having openings or apertures 30 in the side surface 15 extending through to the internal or central bone graft space 50 that allow for laser engraving of interior surfaces 18 across from the apertures 30. The laser beam is able to pass through the apertures 30 and laser modify internal surfaces 18 located across from the aperture or opening 30. As illustrated, two apertures, openings or windows 30 are on one side wall and on an opposite side wall one aperture 30 is positioned offset so apertures 30 are able to provide access portals for the laser beam.

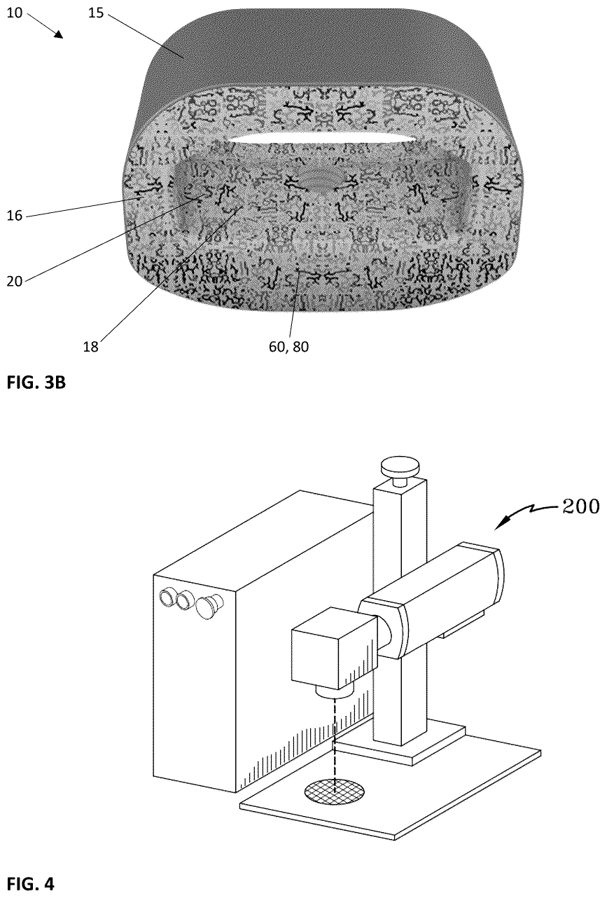

With reference to FIGS. 3A and 3B, a third embodiment implant is illustrated as ALIF 16×32×21 mm with internal facets 20, again angled positively greater than zero, as shown angled at 8 degrees that facilitate laser modification of the internal surface 18 and external surfaces 14, 16 respectively. As shown, the body structure also has openings or windows 30 on the side surface 15.

FIG. 4 is a fourth exemplary embodiment implant device 10 illustrating internal surfaces 18 that are formed with facet or inclination features 20.

With reference to FIG. 4, an exemplary laser engraving machine 200 is illustrated that can be used to form the laser modified network 80. The laser modification can be made either by moving the laser 200 about the exterior surface 14, 15, 16 of the implant device 10 or the implant device 10 can be moved relative to the laser 200.

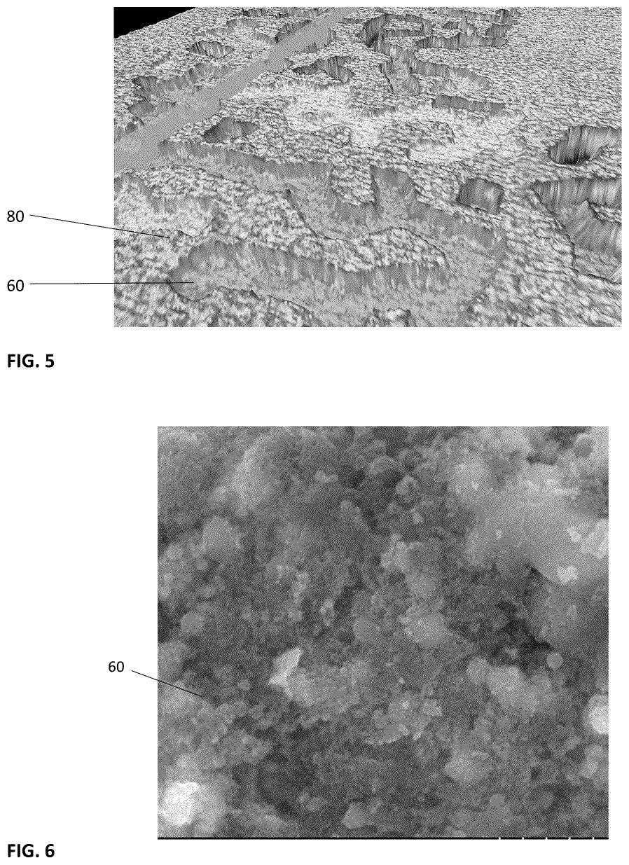

FIG. 5 illustrates the appearance of the laser modified or engraved network 80. The laser modified network 80 can be in either in an organized uniform pattern or a random non-uniform pattern throughout the exterior and/or interior surfaces of the implant 10. The laser modified network 80 creates an improved osteoinductive effect at the surface of the implant device 10. This means that the formation of new bone, once implanted into the patient, can be accelerated and the network provides features that help assist in providing attachment locations for the new bone formation. This is an important feature that is provided in the current invention and is ideal in that it does not require smooth or flat exterior surfaces to form the network which is effectively engraved or burned into the implant. The network can be created as long as the path of the laser beam is unobstructed.

FIG. 6 is a highly magnified image that shows the nano scale features 60 of the laser modified network 80 in which the entire width of the image shown is only about 2 microns. The nano scale features 60 are only visible through powerful magnification which affords sub-micron resolution. As shown, these nano features 60 exhibit very high surface areas in relation to their size. This large surface area creates advantageous regions to induce and to receive new bone growth. The bone-forming cells attach to these nano features 60 with greater case and affinity than on solid untreated surfaces of the implant 10. The nano scale features 60 are prominences projecting less than 200 nano meters from the one or more bone growth surfaces of the implant 10 only visible through magnification of sub micron resolution exhibiting large surface areas relative to the size of the nano scale prominences 60 configured to enhance and receive new bone growth providing the improved osteoinductive feature and variations of surface features resulting from the manufacturing process and the material composition of the implant device 10. An area of the one or more bone growth surfaces of the implant 10 with the nano scale features or prominences 60 having a surface area greater with no additional volume than the surface area of the non-laser engraved surfaces without the nano scale features or prominences and the nano scale features or prominences are less than 200 nano meters formed by subtraction laser engraving process on the surface of the bone growth surfaces of the implant 10. The bone-forming cells become “activated” to form and remodel new bone through biologic changes in their morphology and chemistry due to their interaction with this unique surface structure. Activation is furthered by cell-cell communication, fostering a tissue-based organization that evolves from a cell-based induction. The nano scale features preferably have a width and a depth of 10 nano meters or greater up to 1000 nano meters. These nano scale features 60 are very small and unlike micro channel laser engraving, the nano scale features 60 can be engraved extremely quickly due to their small size and yet due to the increased surface area, greatly improves cell attachment for new bone growth formation.

The subtractive laser modification or engraving process results in nanometer-level or nano scale features on at least a portion of a surface or surfaces of the implant, the nano scale features 60 creating new bone growth attachment features to enhance osteoinductivity of the spinal implant or orthopedic fusion device. The laser engraved nano scale features are made into a network of features in either a random pattern or an organized pattern. The laser modification is formed by emitting laser beams unobstructed to the surfaces of the implant.

The method of making a spinal implant device or orthopedic device or bone implant device according to the present invention can be made by 3D printing and a post process with a laser modification process resulting in nanometer scale of surface structure that is biologically active in inducing bone growth. Alternatively, the implant body structure can be molded or machined to achieve the desired facet features and body structure. Further, the laser engraved surface results in a nanometer scale structure that is active in bone growth formation. The laser modification results in a nanometer scale surface structure because the heat of the laser does not cause a significant melt at the surface that would remove material from the ablation from the nanometer scale of the structure rather than the laser heating it up so that it sears the surface through melting.

As shown, the exemplary embodiments are merely examples of configurations that can be employed to make the present invention. Any number of types of implants and shapes can be used in this configuration and can be any number of polygonal shapes of various shapes and sizes as long as they are sufficient to support the load between the adjacent vertebral bodies to make a proper implant fusion device.

Variations in the present invention are possible in light of the description of it provided herein. While certain representative embodiments and details have been shown for the purpose of illustrating the subject invention, it will be apparent to those skilled in this art that various changes and modifications can be made therein without departing from the scope of the subject invention. It is, therefore, to be understood that changes can be made in the particular embodiments described which will be within the full intended scope of the invention as defined by the following appended claims.

What is claimed is:

1. An implant device configured to be at least partially in contact with bone on implantation having an improved osteoinductive feature to enhance new bone formation, the implant device comprising:
an implant body structure having a superior or first surface and an inferior or second surface, both surfaces configured to be load bearing upon implantation and one or more exterior side surfaces and one or more interior side surfaces extending between the superior and inferior surfaces; and
one or more facet features extending from the internal side surfaces, each of the facet features being inclined off parallel relative to the exterior side surface or off perpendicular relative to the superior and inferior surfaces at an angle which provides a surface to facilitate laser modification of the facet feature when a laser beam is oriented at an angle perpendicular to the load bearing inferior or superior surface.

2. The implant device of claim 1, wherein the facet features are configured to facilitate laser modification to enhance osteoinductivity and promote bone ingrowth located in an internal or central bone graft space.

3. The implant device of claim 1, wherein at least a portion of the implant body structure is exposed to subtractive laser modification which creates a laser modified network having a random or non-random network of trenches, grooves or recesses having nano scale features or prominences for new bone growth formation to attach.

4. The implant device of claim 3, wherein the facet features are exposed to subtractive laser engraving which creates a laser modified network having a random or non-random network of trenches, grooves or recesses having nano scale features or prominences for new bone growth formation to attach.

5. The implant device of claim 3, wherein the laser modified network includes superior and inferior surfaces in contact with bone on implantation as well as surfaces where new bone growth occurs on the internal surfaces and side surfaces of the device that help secure the device in place after bone fusion occurs.

6. The implant device of claim 2, wherein the facet features are inclined at an angle greater than zero off from parallel relative to the interior side surface or off perpendicular relative to the superior and inferior surfaces.

7. The implant device of claim 3, wherein the implant body structure has one or more apertures in the side surface extending through to the internal or central bone graft space that allow for laser modification of interior surfaces across from the apertures.

8. The implant device of claim 3, wherein the nano scale features are prominences projecting less than 200 nano meters from the one or more bone growth surfaces only visible through magnification of sub micron resolution exhibiting large surface areas relative to the size of the nano scale prominences configured to enhance and receive new bone growth providing the improved osteoinductive feature and variations of surface features resulting from the manufacturing process and the material composition of the implant device.

9. The implant device of claim 8, wherein an area of the one or more bone growth surfaces of the implant with the nano scale features or prominences having a surface area greater with no additional volume than the surface area of the non-laser modified surfaces without the nano scale features or prominences and the nano scale features or prominences are less than 200 nano meters formed by subtraction laser modification process on the surface of the bone growth surfaces of the implant.

10. The implant device of claim 1, wherein the inferior and superior surfaces are exposed to subtractive laser modification which creates a laser modified network having a random or non-random network of trenches, grooves or recesses having nano scale features or prominences for new bone growth formation to attach.

11. The implant device of claim 9, wherein the laser modified network when having a non-random network is an organized pattern.

12. The implant device of claim 11, wherein the laser modified network is formed by emitting laser beams unobstructed to surfaces within the path of the laser beams.

13. The implant device of claim 1, wherein the laser modification is made by laser engraving.

14. The implant device of claim 1, wherein the laser modification is made by laser etching.

15. A method of making an implant device configured to be at least partially in contact with bone on implantation having an improved osteoinductive feature to enhance new bone formation, comprises the steps of:
providing an implant body structure, the implant body structure having a superior or first surface and an inferior or second surface, both surfaces configured to be load bearing upon implantation and one or more exterior side surfaces and one or more interior side surfaces extending between the superior and inferior surfaces;
one or more facet features extending from the internal side surfaces, each of the facet features being inclined off parallel relative to the exterior side surface or off perpendicular relative to the superior and inferior surfaces at an angle which provides a surface to facilitate laser modification along the facet feature when a laser beam is oriented at an angle perpendicular to the load bearing inferior or superior surface; and
wherein the implant body structure is stationary and a laser moves about the implant body structure to create a laser modified network or wherein a laser is stationary and the implant body structure moves relative to the laser to create a laser modified network.

16. The method of making a spinal implant device or orthopedic device or bone implant device of claim 15, further comprises the steps of:
laser modifying on at least a portion of an exterior surface or surfaces of the implant body structure to create the laser modified network, the laser modified network creating new bone growth attachment features to enhance osteoinductivity of the spinal implant fusion device.

17. The method of making a spinal implant device or orthopedic device or bone implant device of claim 16, wherein the laser modified network is made into a network of features in either a random pattern or an organized pattern.

18. The method of making a spinal implant device or orthopedic device or bone implant device of claim 17, wherein the laser modification is formed by emitting laser beams unobstructed to the exterior surfaces and the interior side surfaces have one or more facet features.

19. The method of making a spinal implant device or orthopedic device or bone implant device of claim 18, further comprises the step of:
moving a laser about the implant body structure to create the laser modified network.

20. The method of making a spinal implant device or orthopedic device or bone implant device of claim 18, further comprises the step of:
moving the implant body structure about a laser to create the laser modified network.
